(19) United States
(12) Patent Application Publication
(10) Pub. No.: US 2026/0327663 A1
(43) Pub. Date: Oct. 22, 2026

(54) Production of PHA Using Petroleum By-Products

(71) Applicant: DANIMER IPCO, LLC, Bainbridge, GA (US)

(72) Inventors: Isao Noda, Bainbridge, GA (US); Carol G. Leggett, Whigham, GA (US); Phillip Van Trump, Decatur, GA (US)

(21) Appl. No.: 19/491,506

(22) PCT Filed: Jun. 24, 2024
(86) PCT No.: PCT/US2024/035251

§ 371 Date: Dec. 9, 2025

Related U.S. Application Data
(60) Provisional application No. 63/509,819, filed on Jun. 23, 2023.

(51) Int. Cl. C12P 7/625; C12N 1/20; C12N 1/26; C12R 1/01

(52) U.S. Cl. CPC C12P 7/625; C12N 1/20; C12N 1/26; C12R 2001/01

(57) ABSTRACT

The present disclosure provides a method for producing a poly(hydroxyalkanoate) by fermenting a petroleum-derived carbon source with biomass. The petroleum-derived carbon source is made up of: (1) a mixture of petroleum fractions, wherein at least 90 weight percent of the petroleum fractions have a boiling point of 400 °C or greater, or (2) at least 5 weight percent of at least one polycyclic aromatic compound.

### FIELD

This disclosure relates to biodegradable polymers. More particularly, this disclosure relates to methods for producing biodegradable polymers polyhydroxyalkanoates using petroleum-derived carbon sources as a feedstock.

### BACKGROUND

Polyhydroxyalkanoates (PHAs) are a class of biobased polymers of increasing commercial interest, which are also biodegradable and/or compostable.

Polyhydroxyalkanoates are most commonly produced through bio-fermentation in which a carbon source (i.e., a food source) is converted by bacteria or other microbes into the desired polyhydroxyalkanoates. The carbon sources which are used in this bio-fermentation are typically derived from plant oils, hemicellulose, animal oils, sugars, and/or residue from distillation of vegetable oils.

While these carbon sources have been adequate thus far, it would be desirable to provide alternative carbon sources which could be used in the production of polyhydroxyalkanoates, as commercial interest in polyhydroxyalkanoates grows over time.

Accordingly, it would be desirable to produce polyhydroxyalkanoates while utilizing new carbon sources which are otherwise of relatively low commercial value and/or carbon sources which are otherwise deemed hazardous or environmentally unfriendly. For instance, it would be desirable if petroleum by-products which are otherwise undesirable or of low commercial value could be utilized for the production of polyhydroxyalkanoates.

### SUMMARY

The above and other needs are met by a method for producing a poly(hydroxyalkanoate) using a petroleum by-product as a carbon source, according to the present disclosure.

In a first aspect, the present disclosure provides a method for producing a poly(hydroxyalkanoate). In one embodiment, the method includes a first step of mixing a biomass comprising bacteria with a petroleum-derived carbon source. The method also includes a step of fermenting the biomass and the carbon source at a temperature from about 25 °C to about 35 °C for a period of at least 24 hours so that the bacteria of the biomass consume at least a portion of the carbon source and synthesize at least one poly(hydroxyalkanoate). The method further includes a step of separating the at least one poly(hydroxyalkanoate) from the biomass.

According to the present disclosure, the petroleum-derived carbon source is made up of: (1) a mixture of petroleum fractions, wherein at least 90 weight percent of the petroleum fractions have a boiling point of 400 °C or greater as determined by ASTM D7169, or (2) at least 5 weight percent of at least one polycyclic aromatic compound. For instance, the petroleum-derived carbon source may in some instances be made of certain grades of mineral oils, paraffins, pyrolysis oil, and mixtures thereof. The petroleum-derived carbon source may also be made of polycyclic aromatic compounds such as naphthalene and/or anthracene.

In certain preferred embodiments, the petroleum-derived carbon source is made up of a mixture of petroleum fractions and at least 50 weight percent of the petroleum fractions of the carbon source have a boiling point of 470 °C or greater as determined by ASTM D7169.

In certain embodiments, the biomass preferably includes bacteria selected from the group consisting of Cupriavidus sp., Pseudomonas sp., Escherichia sp., and mixtures thereof.

In some instances, the bacteria of the biomass and the carbon source are preferably mixed at a weight ratio of about 50 to about 99 parts of the carbon source to 1 part of the bacteria.

According to certain embodiments, the biomass and the carbon source are preferably fermented at a pH from about 5 to about 9, more preferably from about 6 to about 8, and still more preferably from about 6.5 to about 7.5.

In certain embodiments, the at least one poly(hydroxyalkanoate) preferably has a weight average molecular weight of at least 1000 daltons, as determined by ASTM 5296-19. More preferably, the at least one poly(hydroxyalkanoate) has a weight average molecular weight from about 1,000 to about 3,000,000 daltons, as determined by ASTM 5296-19. Still more preferably, the at least one poly(hydroxyalkanoate) has a weight average molecular weight from about 50,000 to about 500,000 daltons, as determined by ASTM 5296-19.

According to certain embodiments, the at least one poly(hydroxyalkanoate) preferably includes a homopolymer. For instance, the at least one poly(hydroxyalkanoate) may include poly-3-hydroxybutyrate.

In other embodiments, the at least one poly(hydroxyalkanoate) preferably includes a poly(hydroxyalkanoate) copolymer. For instance, the poly(hydroxyalkanoate) may include poly-3-hydroxybutyrate-co-3-hydroxyhexanoate (“P(3HB-co-3HHx)”).

In accordance with some embodiments, the poly(hydroxyalkanoate) copolymer preferably includes at least 87 mole percent monomer repeat units of hydroxybutyrate (such as 3-hydroxybutyrate or 4-hydroxybutyrate) and from about 2 to about 13 mole percent monomer residues of a second hydroxyalkanoate having from 5 to 12 carbon atoms.

In still other embodiments, the poly(hydroxyalkanoate) preferably includes a poly(hydroxyalkanoate) terpolymer. For instance, the poly(hydroxyalkanoate) terpolymer may include from about 75 to about 99.9 mole percent monomer repeat units of 3-hydroxybutyrate, from about 0.1 to about 25 mole percent monomer repeat units of 3-hydroxyhexanoate, and from about 0.1 to about 25 mole percent monomer repeat units of a third 3-hydoxyalkanoate having from 5 to 12 carbon atoms.

In some instances, the carbon source is preferably made up of at least 5 weight percent of at least one polycyclic aromatic compound.

In certain embodiments, the at least one poly(hydroxyalkanoate) synthesized from the one polycyclic aromatic compound includes a polymer backbone having at least one aromatic or polyaromatic moiety incorporated into the polymer backbone. More preferably, the at least one poly(hydroxyalkanoate) includes a polymer backbone having at least one anthracene moiety incorporated into the polymer backbone.

### BRIEF DESCRIPTION OF THE DRAWINGS

Further advantages of the invention are apparent by reference to the detailed description when considered in conjunction with the figures, which are not to scale so as to more clearly show the details, wherein like reference numbers indicate like elements throughout the several views, and wherein:

FIG. 1 is a graph illustrating boiling range distribution for a mineral oil for use in accordance with one embodiment of the present disclosure;

FIG. 2 is a graph illustrating boiling range distribution for a paraffin for use in accordance with one embodiment of the present disclosure; and

FIG. 3 is a NMR spectra of a polyhydroxyalkanoate synthesized in accordance with one embodiment of the present disclosure.

### DETAILED DESCRIPTION

The present disclosure provides a method for producing a poly(hydroxyalkanoate) using a petroleum by-product as an alternative carbon source. Not all organic materials can be digested by bacteria. Further, even if a carbon source is digestible, not all carbon sources are suitable for production of polyhydroxyalkanoates. However, it has surprisingly been found that according to the present disclosure that certain petroleum-derived carbon sources may be used as carbon sources for biological production of poly(hydroxyalkanoates).

In general, the method comprises a first step of mixing a biomass comprising bacteria with a petroleum-derived carbon source. The biomass and the carbon source are then fermented so that that the bacteria of the biomass consume at least a portion of the carbon source and synthesize at least one poly(hydroxyalkanoate). The at least one poly(hydroxyalkanoate) is then separated from the biomass.

Conventionally, the biofermentation of poly(hydroxyalkanoate) polymers is carried out using carbon sources which are agricultural or food-based products. For instance, plant oils, hemicellulose, animal oils, sugars, and/or residue from distillation of vegetable oils are commonly used as a carbon source when producing poly(hydroxyalkanoate) polymers.

According to the present disclosure, however, a petroleum-derived carbon source is used rather than an agricultural or food-based carbon source.

In particular, the petroleum-derived carbon source generally comprises either: (1) a mixture of petroleum fractions, wherein at least 90 weight percent of the petroleum fractions have a boiling point of 400 °C or greater as determined by ASTM D7169, or (2) at least 5 weight percent of at least one polycyclic aromatic compound.

For instance, in some embodiments, the petroleum-derived carbon source may comprise certain grades of mineral oils, paraffins, pyrolysis oil, and mixtures thereof, wherein at least 90 weight percent of the fractions of these carbon sources have a boiling point of 400 °C or greater as determined by ASTM D7169.

For certain embodiments, the petroleum-derived carbon source more preferably comprises a mixture of petroleum fractions, wherein at least 50 weight percent of the petroleum fractions of the carbon source have a boiling point of 470 °C or greater as determined by ASTM D7169.

The petroleum-derived carbon source may also comprise polycyclic aromatic compounds. For instance, the carbon source may include naphthalene, anthracene, and/or other, larger polycyclic aromatic compounds. The petroleum-derived carbon source may for instance comprise at least 5 weight percent of at least one polycyclic aromatic compound. In some embodiments, the petroleum-derived carbon source may comprise at least 10 weight percent of at least one polycyclic aromatic compound, at least 20 weight percent of at least one polycyclic aromatic compound, or at least 30 weight percent of at least one polycyclic aromatic compound.

Thus, it has surprisingly been found that, in accordance with the present disclosure, polyhydroxyalkanoates may be produced while utilizing carbon sources which are otherwise of relatively low commercial value and/or carbon sources which are otherwise deemed hazardous or environmentally unfriendly. This is in contrast to the conventional use of agriculturally-sourced carbon sources such as plant oils, hemicellulose, animal oils, sugars, and/or residue from distillation of vegetable oils.

This petroleum-derived carbon source is mixed with biomass comprising bacteria. Generally, the amount of the petroleum-derived carbon source to be mixed with the biomass may vary, depending upon the specific carbon source being used. Typically, the petroleum-derived carbon source and the bacteria of the biomass are mixed at a weight ratio of about 10 to about 99 parts of the carbon source to 1 part of the bacteria. More preferably, the petroleum-derived carbon source and the biomass are mixed at a weight ratio of about 50 to about 99 parts of the carbon source to 1 part of the bacteria, and still more preferably from about 90 to about 99 parts of the carbon source to 1 part of the bacteria.

Typically, the carbon source and the biomass are mixed in a bio fermentation tank or vessel. The biomass and the carbon source are then fermented at a temperature from about 25 °C to about 35 °C for a period of at least 24 hours so that the bacteria of the biomass consume at least a portion of the carbon source and synthesize at least one poly(hydroxyalkanoate).

More preferably, the biomass and the carbon source are then fermented at a temperature from about 25 °C to about 35 °C, and for a period of at least 72 hours.

In general, the biomass and the carbon source are fermented at a pH from about 5 to about 9, more preferably from about 6 to about 8, and still more preferably from about 6.5 to about 7.5.

The biomass includes one or more bacteria species which are capable of synthesizing poly(hydroxyalkanoate) polymers. Suitable species of bacteria which may be included in the biomass include, but are not limited to, bacteria selected from the group consisting of Cupriavidus sp., Pseudomonas sp., Escherichia sp., and mixtures thereof.

During the fermentation process, the bacteria of the biomass at least a portion of the carbon source and synthesize at least one poly(hydroxyalkanoate).

Typically, the at least one poly(hydroxyalkanoate) synthesized has a weight average molecular weight of at least 1000 daltons, as determined by ASTM 5296-19. More preferably, the at least one poly(hydroxyalkanoate) has a weight average molecular weight from about 5000 to about 3,000,000 daltons, as determined by ASTM 5296-19. Still more preferably, the at least one poly(hydroxyalkanoate) has a weight average molecular weight from about 50,000 to about 500,000 daltons, as determined by ASTM 5296-19.

In various embodiments, the at least one poly(hydroxyalkanoate) synthesized by the biomass may be a homopolymer, a copolymer, a terpolymer, or a mixture of the foregoing.

In certain embodiments, the at least one poly(hydroxyalkanoate) may comprise a homopolymer. For instance, the at least one poly(hydroxyalkanoate) may include poly-3-hydroxybutyrate.

In other embodiments, the at least one poly(hydroxyalkanoate) may comprise a poly(hydroxyalkanoate) copolymer. For instance, the poly(hydroxyalkanoate) may include poly-3-hydroxybutyrate-co-3-hydroxyhexanoate (“P(3HB-co-3HHx)”).

In accordance with some embodiments, the poly(hydroxyalkanoate) copolymer preferably includes at least 87 mole percent monomer repeat units of hydroxybutyrate (such as 3-hydroxybutyrate or 4-hydroxybutyrate) and from about 2 to about 13 mole percent monomer residues of a second hydroxyalkanoate having from 5 to 12 carbon atoms.

In still other embodiments, the poly(hydroxyalkanoate) may comprise a poly(hydroxyalkanoate) terpolymer. For instance, the poly(hydroxyalkanoate) terpolymer may include from about 75 to about 99.9 mole percent monomer repeat units of 3-hydroxybutyrate, from about 0.1 to about 25 mole percent monomer repeat units of 3-hydroxyhexanoate, and from about 0.1 to about 25 mole percent monomer repeat units of a third 3-hydoxyalkanoate having from 5 to 12 carbon atoms.

Moreover, in some embodiments in which the carbon source includes at least one polycyclic aromatic compound, it has also surprisingly been found that the at least one poly(hydroxyalkanoate) synthesized from the carbon source may comprise a polymer backbone having at least one aromatic or polyaromatic moiety incorporated into the polymer backbone. For example, in some instances, the at least one poly(hydroxyalkanoate) may include at least one anthracene moiety incorporated into its polymer backbone.

Finally, according to the present disclosure, the at least one poly(hydroxyalkanoate) is separated from the biomass. This separation may involve one or a series of steps and may include the addition of various enzymatic agents such as a lysing agent to disrupt the cell walls of the bacteria, an endonuclease to cleave polynucleotide chains in the bacteria, and/or a peptidase to degrade proteins within the bacteria. A surfactant or detergent may also be used to facilitate separation of the poly(hydroxyalkanoate) from the biomass.

### EXAMPLES

The following non-limiting examples illustrate various additional aspects of the invention. Unless otherwise indicated, temperatures are in degrees Celsius, and percentages are by weight, based on the dry weight of the formulation.

In this series of experiments, a variety of carbon source materials were tested to determine if bacteria could metabolize the carbon source and use it to synthesize a poly(hydroxyalkanoate)

As an initial step, a series of stock solutions were prepared, as follows:

### Solution 1 Buffered Production Media 2 (BPM2)

| Component | Concentration (grams/Liter) | |
|---|---|---|
| Reverse Osmosis (RO) water | 850 | mL |
| NaOH pellet | 1.33 | grams |
| KH2PO4 (anhydrous) | 9.12 | grams |
| MgSO4*7H2O | 2 | grams |
| Trace A (what is this?) | 5 | mL |

| | |
|---|---|
| 1N NaOH | q.s. pH to 6.8 |

### Solution 2 (NH4)2SO4

| Component | Amount |
|---|---|
| RO water | 90 ml |
| (NH4)2SO4 | 12.00 grams |

### Solution 3A Refined, Bleached & Deodorized (RBD) Canola Oil

| Component | Amount |
|---|---|
| RBD Canola Oil | 50 mL |

### Solution 3B Anthracene

| Component | Amount |
|---|---|
| Anthracene | 1.432 grams |
| Methanol | 100 mL |

### Solution 3C Naphthalene

| Component | Amount |
|---|---|
| Naphthalene | 6.030 grams |
| Methanol | 100 mL |

### Solution 3D Mineral Oil Plus 3% Gum Arabic Emulsifier

| Component | Amount |
|---|---|
| Mineral Oil | 50 mL |
| Gum Arabic | 1.5 mL |

### Solution 3E Paraffin Plus 3% Gum Arabic Emulsifier

| Component | Amount |
|---|---|
| Paraffin | 50 mL |
| Gum Arabic | 1.5 mL |
| RO water | 50 mL |

### Solution 3F Mineral Oil Without Emulsifier

| Component | Amount |
|---|---|
| Mineral Oil | 50 mL |
| Gum Arabic | 1.5 mL |

### Solution 3G Paraffin Without Emulsifier

| Component | Amount |
|---|---|
| Paraffin | 50 mL |

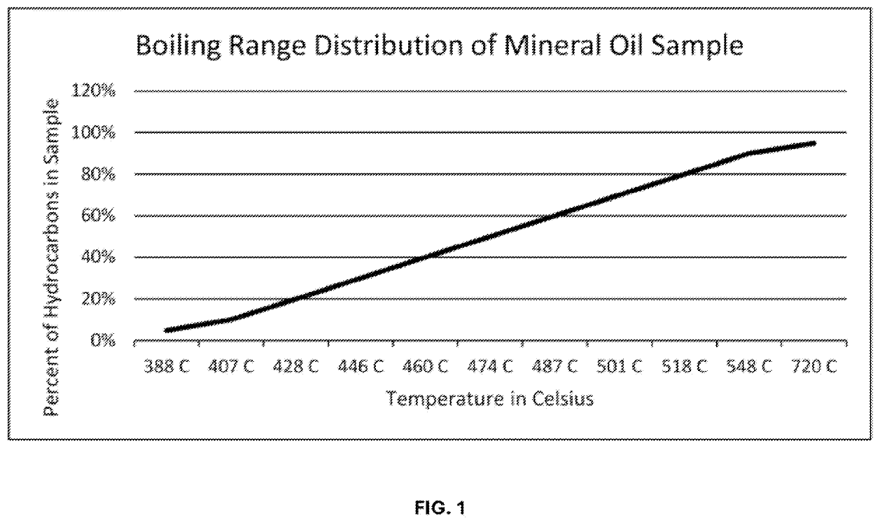

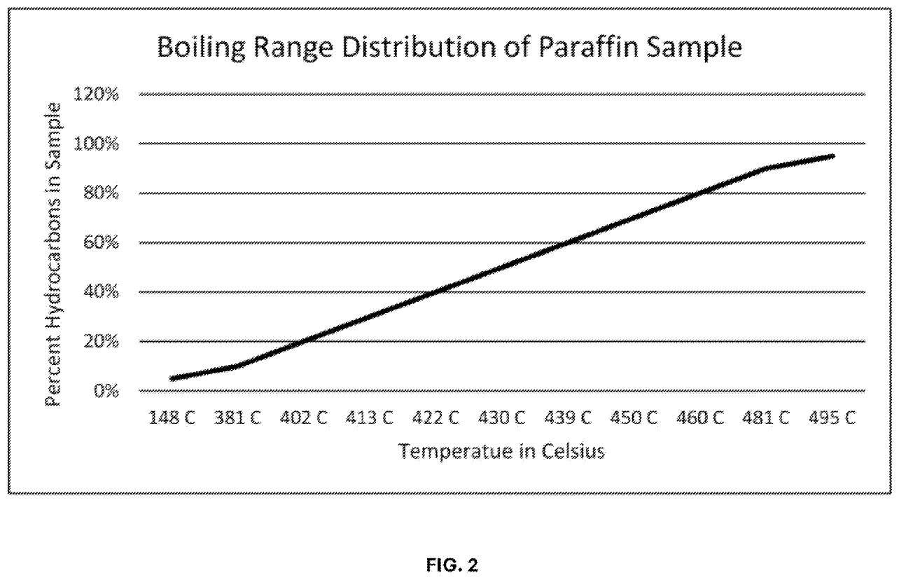

Samples of the mineral oil and paraffin used in solutions 3D-3G were also submitted to a petroleum laboratory (SGS) to determine the boiling range distribution of carbon residues within these compounds, according to ASTM D7169. The boiling range distribution data for the mineral oil and the paraffin are shown in FIGS. 1 and 2 respectively.

Next, a series of shake flasks containing production media were prepared from the aforementioned stock solutions. The pH of each shake flask was approximately 6.8. The composition for each of the shake flasks was as follows:

### BPM2 15% Canola Control Shake Flask #1A

| Component | Amount | |
|---|---|---|
| Solution 2 | 0.8 | mL |
| RBD Canola Oil (3A) | 11.6 | mL |
| BPM2 | 63.75 | mL |

### BPM2 5% Canola Control Shake Flask #1B

| Component | Amount |
|---|---|
| Solution 2 | 0.8 mL |
| RBD Canola Oil (3A) | 3.8 mL |
| BPM2 | 67.5 mL |

### BPM2 5% Anthracene Shake Flask #2A

| Component | Amount |
|---|---|
| Solution 2 | 0.8 mL |
| Anthracene (3B) | 3.8 mL |
| BPM2 | 67.5 mL |

### BPM2 5% Anthracene Shake Flask #2B

| Component | Amount |
|---|---|
| Solution 2 | 0.8 mL |
| Anthracene (3B) | 3.8 mL |
| BPM2 | 67.5 mL |

### BPM2 5% Naphthalene Shake Flask #3A

| Component | Amount |
|---|---|
| Solution 2 | 0.8 mL |
| Naphthalene (3C) | 3.8 mL |
| BPM2 | 67.5 mL |

### BPM2 5% Naphthalene Shake Flask #3B

| Component | Amount |
|---|---|
| Solution 2 | 0.8 mL |
| Naphthalene (3C) | 3.8 mL |
| BPM2 | 67.5 mL |

### BPM2 15% Mineral Oil Plus Gum Arabic Shake Flask #4A

| Component | Amount | |
|---|---|---|
| Solution 2 | 0.8 | mL |
| Mineral Oil/Gum Arabic (3D) | 11.6 | mL |
| BPM2 | 63.75 | mL |

### BPM2 15% Mineral Oil Plus Gum Arabic Shake Flask #4B

| Component | Amount | |
|---|---|---|
| Solution 2 | 0.8 | mL |
| Mineral Oil/Gum Arabic (3D) | 11.6 | mL |
| BPM2 | 63.75 | mL |

### BPM2 15% Paraffin Plus Gum Arabic Shake Flask #5A

| Component | Amount | |
|---|---|---|
| Solution 2 | 0.8 | mL |
| Paraffin/Gum Arabic (3E) | 11.6 | mL |
| BPM2 | 63.75 | mL |

### BPM2 15% Paraffin Plus Gum Arabic Shake Flask #5B

| Component | Amount | |
|---|---|---|
| Solution 2 | 0.8 | mL |
| Paraffin/Gum Arabic (3E) | 11.6 | mL |
| BPM2 | 63.75 | mL |

### BPM2 15% Mineral Oil Shake Flask #6A

| Component | Amount | |
|---|---|---|
| Solution 2 | 0.8 | mL |
| Mineral Oil (3F) | 11.6 | mL |
| BPM2 | 63.75 | mL |

### BPM2 15% Mineral Oil Shake Flask #6B

| Component | Amount | |
|---|---|---|
| Solution 2 | 0.8 | mL |
| Mineral Oil (3F) | 11.6 | mL |
| BPM2 | 63.75 | mL |

### BPM2 15% Paraffin Shake Flask #7A

| Component | Amount | |
|---|---|---|
| Solution 2 | 0.8 | mL |
| Paraffin (3G) | 11.6 | mL |
| BPM2 | 63.75 | mL |

### BPM2 15% Paraffin Shake Flask #7B

| Component | Amount | |
|---|---|---|
| Solution 2 | 0.8 | mL |
| Paraffin (3G) | 11.6 | mL |
| BPM2 | 63.75 | mL |

It was previously determined that bacteria could not metabolize either methanol solvent or gum Arabic emulsifier in order to synthesize poly(hydroxyalkanoate). Thus, the production media composition in each shake flask included only a single possible carbon source for poly(hydroxyalkanoate) synthesis.

Separately, 60 milliliters of yeast, wheat peptone, and fructose (YWF) media in a 6-baffled 250 mL flask were inoculated with 10 mL of Cupriavidus necator H16 culture and allowed to incubate at 30 °C; 300 rpm for 18 hours to produce a bacterial inoculum.

Next approximately 9 mL of the Cupriavidus necator bacterial inoculum was then added to the carbon source mixture in each of the shake flasks. The shake flasks were then incubated at 300 rpm in a rotary shaker at 30 °C for 72 hours.

To recover any poly(hydroxyalkanoate) produced during the incubation, the cultures from each shake flask were each centrifuged at 10,000×g for 5 minutes in a 50 mL centrifuge tube. The supernatant was decanted, and the pellet was resuspended in 50 mL RO water using a vortex. The pellet was processed using cocktail A and SeBrite (a protease available from Specialty Enzymes) in a 250 mL six-baffled flask at 37 °C, and at 58 °C respectively. Cocktail A was made up of a mixture of 5.5 mL of Tween 20 surfactant, 5.5 mL of Tween 80 surfactant, 0.66 grams lysozyme, and 44 microliters (L) of nuclease. Hydrogen peroxide was added, and the pellets were washed twice with RO water and ethanol. After drying (benchtop) the recovered material was submitted for analysis to determine (1) the presence or absence of poly(hydroxyalkanoate) using NMR and/or FTIR analysis and (2) to determine the mole percentage of hydroxyhexanoate monomer in any poly-3-hydroxybutyrate-co-3-hydroxyhexanoate (“P(3HB-co-3HHx)”) copolymer using NMR analysis.

The results are as follow:

| Flask | | | NMR analysis of |
|---|---|---|---|
| No. | Carbon Source | PHA Present? | C4C6 copolymer |
| 1A | 15% Canola Control | Yes | 3.3 mole % C6 |
| 1B | 5% Canola Control | Yes | 3.7 mole % C6 |
| 2A | 5% Anthracene | Yes | 7 mole % C6 |
| 2B | 5% Anthracene | Yes | 9.3 mole % C6 |
| 3A | 5% Naphthalene | Yes | 8.3 mole % C6 |
| 3B | 5% Naphthalene | Insufficient | Insufficient |
| | | Sample | Sample |
| 4A | 15% Mineral Oil & Gum | Yes | 2.3 mole % C6 |
| | Arabic | | |
| 4B | 15% Mineral Oil & Gum | Yes | Inconclusive |
| | Arabic | | |
| 5A | 15% Paraffin & Gum Arabic | Yes | 11.3 mole % C6 |
| 5B | 15% Paraffin & Gum Arabic | Yes | 11.3 mole % C6 |
| 6A | 15% Mineral Oil | Insufficient | Insufficient |
| | | Sample | Sample |
| 6B | 15% Mineral Oil | Yes | Trace |
| 7A | 15% Paraffin | Yes | 13.3 mole % C6 |
| 7B | 15% Paraffin | Yes | 9 mole % C6 |

From the above results, the presence of poly(hydroxyalkanoate) in the culture broth that contained the polyaromatics, anthracene and naphthalene as the sole source of carbon, confirms the utilization of these polyaromatic compounds in the production of polyhydroxyalkanoates, including poly(3-hydroxybutyrate) co-poly(3-hydroxyhexanoate) copolymer. Furthermore, this experiment confirms the use of long petroleum-based hydrocarbon chains such as those found in mineral oil and paraffin, in the production of polyhydroxyalkanoates, including the comonomer poly(3-hydroxybutyrate) co-poly(3-hydroxyhexanoate).

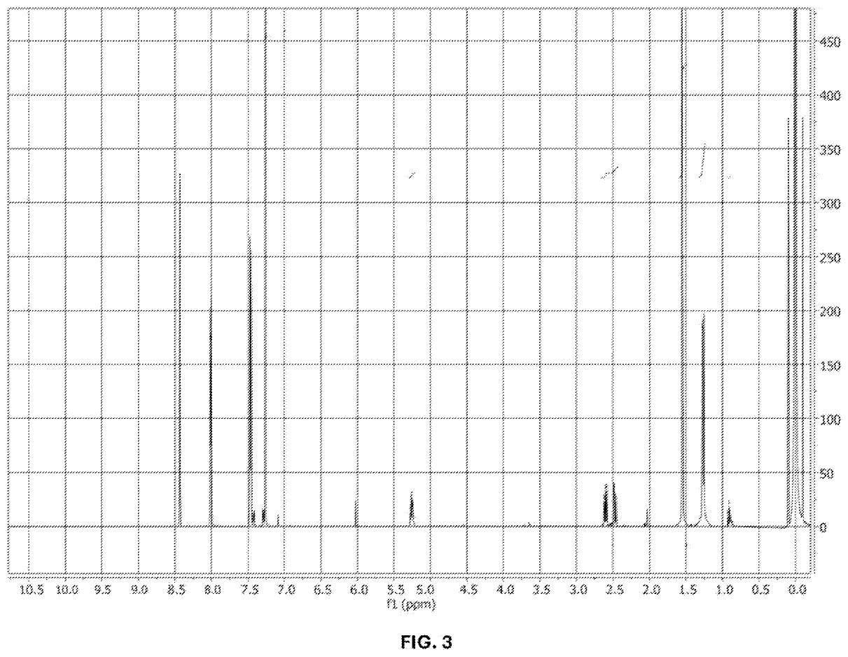

Finally, the polyhydroxyalkanoate samples from the anthracene and naphthalene shake flasks were also submitted to NMR analysis to verify the presence of anthracene and/or naphthalene within the polyhydroxyalkanoate molecule. The samples from the anthracene shake flasks were found to have incorporated an aromatic anthracene ring into the polyhydroxyalkanoate polymer chain. This is illustrated in the NMR spectra shown in FIG. 3 in which the peaks at approximately 7, 8, and 8.5 indicate the presence of an anthracene moiety in the polymer backbone of the polyhydroxyalkanoate. There was no indication that naphthalene was similarly incorporated into the polyhydroxyalkanoate polymer chain.

### EMBODIMENTS

The present disclosure is also further illustrated by the following embodiments:

Embodiment 1. A method for producing a poly(hydroxyalkanoate) comprising the steps of:
mixing a biomass comprising bacteria with a petroleum-derived carbon source;
fermenting the biomass and the carbon source at a temperature from about 25 °C to about 35 °C for a period of at least 24 hours so that the bacteria of the biomass consume at least a portion of the carbon source and synthesize at least one poly(hydroxyalkanoate); and
separating the at least one poly(hydroxyalkanoate) from the biomass,
wherein the petroleum-derived carbon source comprises: (1) a mixture of petroleum fractions, wherein at least 90 weight percent of the petroleum fractions have a boiling point of 400 °C or greater as determined by ASTM D7169, or (2) at least 5 weight percent of at least one polycyclic aromatic compound.

Embodiment 2. The method of Embodiment 1, wherein the petroleum-derived carbon source comprises a mixture of petroleum fractions and at least 50 weight percent of the petroleum fractions of the carbon source have a boiling point of 470 °C or greater as determined by ASTM D7169.

Embodiment 3. The method of Embodiment 1 or 2, wherein the biomass comprises bacteria selected from the group consisting of Cupriavidus sp., Pseudomonas sp., Escherichia sp., and mixtures thereof.

Embodiment 4. The method of any of the preceding Embodiments, wherein the bacteria of the biomass and the carbon source are mixed at a weight ratio of about 50 to about 99 parts of the carbon source to 1 part of the bacteria.

Embodiment 5. The method of any of the preceding Embodiments, wherein the biomass and the carbon source are fermented at a pH from about 6.5 to about 7.5.

Embodiment 6. The method of any of the preceding Embodiments, wherein the at least one poly(hydroxyalkanoate) has a weight average molecular weight of at least 1,000 daltons, as determined by ASTM 5296-19, preferably from about 1,000 to about 3,000,000 daltons, and more preferably from about 50,000 to about 500,000 daltons,

Embodiment 7. The method of any of the preceding Embodiments, wherein the at least one poly(hydroxyalkanoate) comprises a homopolymer.

Embodiment 8. The method of any of the preceding Embodiments, wherein the at least one poly(hydroxyalkanoate) comprises poly-3-hydroxybutyrate.

Embodiment 9. The method of any of the preceding Embodiments, wherein the at least one poly(hydroxyalkanoate) comprises a poly(hydroxyalkanoate) copolymer.

Embodiment 10. The method of Embodiment 9, wherein the poly(hydroxyalkanoate) copolymer comprises at least 87 mole percent monomer repeat units of hydroxybutyrate (such as 3-hydroxybutyrate or 4-hydroxybutyrate) and from about 2 to about 13 mole percent monomer residues of a second hydroxyalkanoate having from 5 to 12 carbon atoms.

Embodiment 11. The method of any of the preceding Embodiments, wherein the poly(hydroxyalkanoate) comprises a poly(hydroxyalkanoate) terpolymer.

Embodiment 12. The method of Embodiment 11, wherein the poly(hydroxyalkanoate) terpolymer comprises from about 75 to about 99.9 mole percent monomer repeat units of 3-hydroxybutyrate, from about 0.1 to about 25 mole percent monomer repeat units of 3-hydroxyhexanoate, and from about 0.1 to about 25 mole percent monomer repeat units of a third 3-hydoxyalkanoate having from 5 to 12 carbon atoms.

Embodiment 13. The method of any of the preceding Embodiments, wherein the carbon source comprises at least one polycyclic aromatic compound.

Embodiment 14. The method of Embodiment 13, wherein the at least one poly(hydroxyalkanoate) comprises a poly(hydroxyalkanoate) having a polymer backbone with at least one aromatic or polyaromatic moiety incorporated into the polymer backbone.

Embodiment 15. The method of Embodiment 13, wherein the at least one poly(hydroxyalkanoate) comprises a poly(hydroxyalkanoate) having a polymer backbone with at least one anthracene moiety incorporated into the polymer backbone.

The foregoing description of preferred embodiments for this disclosure has been presented for purposes of illustration and description. It is not intended to be exhaustive or to limit the disclosure to the precise form disclosed. Obvious modifications or variations are possible in light of the above teachings. The embodiments are chosen and described in an effort to provide the best illustrations of the principles of the disclosure and its practical application, and to thereby enable one of ordinary skill in the art to utilize the disclosure in various embodiments and with various modifications as are suited to the particular use contemplated.

1. A method for producing a poly(hydroxyalkanoate) comprising the steps of:
mixing a biomass comprising bacteria with a petroleum-derived carbon source;
fermenting the biomass and the carbon source at a temperature from 25 °C to 35 °C for a period of at least 24 hours so that the bacteria of the biomass consume at least a portion of the carbon source and synthesize at least one poly(hydroxyalkanoate); and
separating the at least one poly(hydroxyalkanoate) from the biomass,
wherein the petroleum-derived carbon source comprises: (1) a mixture of petroleum fractions,
wherein at least 90 weight percent of the petroleum fractions have a boiling point of 400 °C or greater as determined by ASTM D7169, and wherein the petroleum fractions are mineral oils, paraffins, or mixtures thereof, or (2) at least 5 weight percent of at least one polycyclic aromatic compound comprising anthracene.

2. The method of claim 1, wherein the petroleum-derived carbon source comprises a mixture of petroleum fractions and at least 50 weight percent of the petroleum fractions of the carbon source have a boiling point of 470 °C or greater as determined by ASTM D7169.

3. The method of claim 1, wherein the biomass comprises bacteria selected from the group consisting of Cupriavidus sp., Pseudomonas sp., Escherichia sp., and mixtures thereof.

4. The method of claim 1, wherein the bacteria of the biomass and the carbon source are mixed at a weight ratio of 50 to 99 parts of the carbon source to 1 part of the bacteria.

5. The method of claim 1, wherein the biomass and the carbon source are fermented at a pH from 6.5 to 7.5.

6. The method of claim 1, wherein the at least one poly(hydroxyalkanoate) has a weight average molecular weight of at least 1,000 daltons, as determined by ASTM 5296-19.

7. The method of claim 1, wherein the at least one poly(hydroxyalkanoate) comprises a homopolymer.

8. The method of claim 1, wherein the at least one poly(hydroxyalkanoate) comprises poly-3-hydroxybutyrate.

9. The method of claim 1, wherein the at least one poly(hydroxyalkanoate) comprises a poly(hydroxyalkanoate) copolymer.

10. The method of claim 9, wherein the poly(hydroxyalkanoate) copolymer comprises at least 87 mole percent monomer repeat units of hydroxybutyrate and from 2 to 13 mole percent monomer residues of a second hydroxyalkanoate having from 5 to 12 carbon atoms.

11. The method of claim 1, wherein the poly(hydroxyalkanoate) comprises a poly(hydroxyalkanoate) terpolymer.

12. The method of claim 11, wherein the poly(hydroxyalkanoate) terpolymer comprises from 75 to 99.9 mole percent monomer repeat units of 3-hydroxybutyrate, from 0.1 to 25 mole percent monomer repeat units of 3-hydroxyhexanoate, and from 0.1 to 25 mole percent monomer repeat units of a third 3-hydoxyalkanoate having from 5 to 12 carbon atoms.

13.-14. (canceled)

15. The method of claim 1, wherein the at least one poly(hydroxyalkanoate) comprises a poly(hydroxyalkanoate) having a polymer backbone with at least one anthracene moiety incorporated into the polymer backbone.

16. The method of claim 1, wherein the petroleum fractions comprise paraffins.

17. The method of claim 1, wherein the carbon source comprises (2) at least 10 weight percent of at least one polycyclic aromatic compound comprising anthracene, wherein the at least one poly(hydroxyalkanoate) comprises a poly(hydroxyalkanoate) having a polymer backbone with at least one anthracene moiety incorporated into the polymer backbone.

18. The method of claim 1, wherein the carbon source comprises (2) at least 20 weight percent of at least one polycyclic aromatic compound comprising anthracene, wherein the at least one poly(hydroxyalkanoate) comprises a poly(hydroxyalkanoate) having a polymer backbone with at least one anthracene moiety incorporated into the polymer backbone.

19. The method of claim 1, wherein the carbon source comprises (2) at least 30 weight percent of at least one polycyclic aromatic compound comprising anthracene, wherein the at least one poly(hydroxyalkanoate) comprises a poly(hydroxyalkanoate) having a polymer backbone with at least one anthracene moiety incorporated into the polymer backbone.

20. The method of claim 1, wherein the at least one poly(hydroxyalkanoate) has a weight average molecular weight of at least from about 1,000 to about 3,000,000 daltons, as determined by ASTM 5296-19.

21. A composition comprising at least one poly(hydroxyalkanoate) having a polymer backbone with at least one anthracene moiety incorporated into the polymer backbone.

22. The composition of claim 21, wherein the at least one poly(hydroxyalkanoate) has a weight average molecular weight of at least from about 1,000 to about 3,000,000 daltons, as determined by ASTM 5296-19.
